SEQUENCE LISTING (2 sequences)

SEQ ID NO: 1 - DNA, 23 bp, Homo sapiens
tgrgcctyartttccccayctgt

SEQ ID NO: 2 - DNA, 200 bp, Homo sapiens
  misc_feature (1)..(200): The sequence  from-1067 to -868 of human NIS promoter with A in ATG of hNIS is designated as +1
taaatgaatgaggcacttatcatgtaccaggctttcgttaccacgtcccatttattcctctgaggcagggtctattttatccttgttacagatggggaaactaaggcccagggaggagcaaagtcttccccaagtatgtacccactcagaacttgagctctgaatgtctcccacccagcttagcccaagagcggggttca

(19) United States
(12) Patent Application Publication
(10) Pub. No.: US 2011/0229878 A1
(43) Pub. Date: Sep. 22, 2011

(54) HUMAN CONSENSUS SODIUM-IODIDE SYMPORTER REPRESSOR (NIS-REPRESSOR) BINDING SITE

(76) Inventors: Kenneth AIN, Lexington, KY (US); Wei Li, Lexington, KY (US)

(21) Appl. No.: 13/044,120

(22) Filed: Mar. 9, 2011

Related U.S. Application Data
(63) Continuation-in-part of application No. 12/724,898, filed on Mar. 16, 2010.

(51) Int. Cl. C12Q 1/68; C07H 21/04; G01N 27/447

(52) U.S. Cl. 435/6.1; 536/23.1; 536/24.5; 204/450

(57) ABSTRACT

The present disclosure relates to a Sodium-Iodide Symporter-repressor (NIS-repressor) binding site (NRBS) consensus sequence consisting of a DNA molecule having the sequence: 5′-T/C(G/A)GCCT(T/C)A(G/A)TTTCCCCA(T/C)CTGT-3.′ The disclosure further relates to methods of screening compounds and other molecules that bind to or inhibits the NIS-repressor or inhibits or interferes with the binding of NIS-repressor to the NIS-repressor binding site.

### RELATED APPLICATIONS

This application is a continuation-in-part of U.S. application Ser. No. 12/724,898, filed Mar. 16, 2010.

### STATEMENT OF GOVERNMENT SUPPORT

This disclosure was made, in part, with support from the Merit Review award program of the U.S. Department of Veterans Affairs and an R01 Grant from the National Cancer Institute of the National Institutes of Health, and the government may have certain rights in this disclosure.

### FIELD OF THE INVENTION

This disclosure relates to a consensus nucleotide sequence found within two kilobases of the 5′ end of fifty-six different genes in the human genome and use of the consensus sequence to screen for compounds and other molecules that inhibit transcription or that inhibit or interfere with transcription repressors or repressor complexes.

### BACKGROUND OF THE INVENTION

Human sodium-iodine symporter (hNIS) is a trans-membrane protein enabling thyrocytes, both benign and malignant, to concentrate iodine; permitting radioiodine to be a unique systemic cytotoxic therapy for metastatic tumors. Unfortunately, when hNIS expression is lost in dedifferentiated thyroid carcinomas, there are no effective systemic cytotoxic agents (Ain 2000).

Previous investigations revealed evidence for an alternative mechanism for loss of hNIS transcription, suggesting presence of a trans-acting repressor of hNIS transcription, termed NIS-repressor (Li, et al. 2007).

Multiple cellular and nuclear factors are reported to be important for hNIS transcription, including: TSH (thyrotropin)/receptor (TSHr) (Riedel, et al. 2001), TTF-1 (Schmitt, et al. 2001), and Pax-8 (Pasca di Magliano, et al. 2000), but there are no clear examples of repressing transcription factors in thyroid cells or thyroid carcinomas. In U.S. application 60/907,881, we showed NIS-repressor as a trans-acting protein binding to a specific region of the proximal hNIS promoter, NIS-repressor binding site (NRBS-P); however its composition was not yet known. We also characterized NIS-repressor and investigated the identities of its components and mechanisms of its activity. This involved defining NRBS-P to a narrower region of hNIS promoter and utilizing it to probe nuclear extract, analyzing the probe-bound proteins with liquid chromatography coupled with tandem mass spectrometry (LC/MS/MS), to characterize NIS-repressor components. The mass spectrometry analysis data demonstrated human PARP-1 (poly(ADP-ribose)polymerase-I) to be a likely component of the NIS-repressor protein complex. Pharmacological inhibition of PARP-1 activity with PJ34, a PARP-1 inhibitor, stimulated endogenous hNIS mRNA levels, providing evidence that PARP-1 acts as a negative regulatory factor for hNIS transcription and is a likely component of the NIS-repressor complex.

Because of its role in inhibiting the transport of iodine into cells, and in particular, into thyroid cancer cells, there is a need to determine the hNIS repressor binding sites, structure and activities so that anti-thyroid cancer therapies can be maximized. Further, there is a need to determine the general applicability of PARP-1 inhibition to alter transcription regulation and the role of the hNIS repressor binding site in transcription regulation in general.

### SUMMARY OF THE INVENTION

One aspect of the invention relates to a sodium iodine symporter (NIS)-repressor binding site (NRBS) consisting of a DNA molecule spanning from −1067 to −868 (SEQ ID NO.: 2). Another aspect of the invention relates to a transcription-repressor binding site consisting of a DNA molecule having the sequence 5′-TG(G/A)GCCT(T/C)A(G/A)TTTCCCCA(T/C)CTGT-3′ (SEQ ID NO.: 1) (NRBS consensus sequence) or a nucleotide sequence that hybridizes to the full length of the complement thereof under high stringency conditions. In certain embodiments of this aspect of the invention, there is provided a vector or expression cassette comprising the consensus sequence operably linked to a promoter sequence which is operably linked to a reporter gene, such as a gene encoding a detectable marker, e.g., a luciferase gene. In certain embodiments, the vector is an adenovirus vector.

Yet another aspect of the invention relates to a method of treating thyroid cancer comprising administering to a patient in need thereof a therapeutically effective amount of a PARP-1 inhibitor and a therapeutically effective amount of radiolabeled iodine. In an other aspect of the invention there is provided a method of treating thyroid cancer in a patient comprising contacting thyroid cancer cells in the patient that express and form a NIS repressor protein complex capable of binding to SEQ ID NO.: 1 or SEQ ID NO.: 2 with a PARP-1 inhibitor, and administering to the cells radiolabeled iodine.

Another aspect of the invention relates to a method of screening molecules or compounds that bind to SEQ ID NO. 1 and inhibit or interfere with transcription, said method comprising (1) contacting the test molecule or compound with a nucleotide sequence comprising SEQ ID NO. 1 and (2) determining whether the test molecule or compound binds to SEQ ID NO. 1. Those test compounds or molecules that bind to SEQ ID NO. 1 may be selected for further testing to determine if they modulate target gene expression.

Another aspect of the invention relates to a method for screening molecules or compounds that interfere with NIS repressor binding to the SEQ ID NO. 1, said method comprising (1) contacting the test molecule or compound in the presence of human NIS repressor with a nucleotide sequence comprising SEQ ID NO. 1 and (2) detecting an alteration in binding of the NIS repressor to SEQ ID NO. 1. Those test compounds or molecules that alter NIS repressor binding to that bind to SEQ ID NO. 1 may be selected for further testing to determine if they modulate target gene expression.

### BRIEF DESCRIPTION OF THE DRAWINGS

FIGS. 1A and B show the results of EMSA analysis followed by SDS electrophoresis to find additional binding sites for NIS-repressor. In FIG. 1A radiolabeled Probe-A, radiolabeled SHIFT-1, radiolabeled SHIFT-2, radiolabeled SHIFT-3 were used in lanes 1 to 3, 4 to 6, 7 to 9, and 10 to 12, respectively. Lanes 1, 4, 7, and 10 contain the respective labeled probes only. KAK1 nuclear extract is included in all other lanes, with lanes 3, 6, 9, and 12 containing 30× unlabeled respective probe. In FIG. 1B, radiolabeled Probe-A, radiolabeled SHIFT-4, and radiolabeled SHIFT-5, are used in lanes 1 to 3, 4 to 6, and 7 to 9, respectively. Lanes 1, 4, and 7 contain the respective hot probes only. KAK1 nuclear extract is included in all other lanes, with lanes 3, 6, and 9 containing 30× unlabeled respective probe. The arrows point to probe-specific bands.

FIGS. 2A and 2B show the results of EMSA analysis followed by SDS gel electrophoresis to define the core sequence for NRBS-D and cross competition of NRBS-D with NRBS-P. FIG. 2A depicts EMSA using KAK1 nuclear extract probed with radiolabeled SHIFT-4 containing NRBS-D in lanes 2 to 15. Unlabeled (30×) SHIFT-4, 4.1, 4.4, 4.2, 4.3, 4.5, 4.6, 4.7, and Probe-A were included in EMSA reactions in lanes 3, 4, 5, 6, 7, 11, 12, 13, and 15, respectively. The unlabeled (60×) annealed double-stranded oligonucleotides ds-411, ds-412, ds-413, and Comp-1 were added to the EMSA reactions in lanes 8, 9, 10, and 14, respectively. Lane 2 had no additional unlabeled competitor, and lane 1 contained radiolabeled SHIFT-4 probe only. In FIG. 2B, KAK1 nuclear extract was probed with radiolabeled Probe-A. The unlabeled 30× Probe-A, 60× annealed double-stranded Comp-1, and 60× annealed double-stranded ds-414 were added in EMSA reactions in lane 3, 4, and 5, respectively. The arrows point to the probe-specific bands.

FIGS. 3A, B and C show the results of a supershift experiment in which antibodies against thyroid-related transcription factors using Cal-62 nuclear extract with a probe that contains NRBS-P (bp −653 to −615) or NRBS-D. Experiments depicted in 3A and 3B were performed using Comp-1 probe while experiments in 3C used SHIFT-414 probe. In all three sections Lane 1 contains probe only with all other lanes containing basal Cal-62 nuclear extract and Lane 3 contains 50× cold respective probes. In 3A, specific antibodies were added to respective lanes as follows: Lane 4, anti-TTF-1; Lane 5, anti-TTF-2 (S-18); Lane 6, anti-Pax8; Lane 7, anti-Sp1; Lane 8, anti-c-Jun; Lane 9, anti-c-Fos; Lane 10, anti-AP2α; and Lane 11, anti-PARP-1. In both 3B and 3C, specific antibodies were added as follows: Lane 4, anti-TTF-2 (S-18); Lane 5, anti-TTF-2 (F-17); and Lane 6, anti-TTF-2 (V-20).

### DETAILED DESCRIPTION

Radioiodine therapy remains the only known effective systemic tumoricidal treatment for thyroid carcinoma. Unfortunately, around 10% of such cancers and most dedifferentiated thyroid cancers fail to concentrate radioiodine consequent to loss of sodium-iodine symporter gene (NIS) expression (Ain 2000; Robbins, et al. 1991). For that reason, efforts to understand the mechanisms of this loss may lead to new treatments to restore NIS expression, permitting effective therapy with radioiodine. Our previous study provided evidence of a trans-active protein factor (complex) suppressing NIS transcription under basal conditions, possibly accounting for loss of human NIS expression in some thyroid cancers. This suggested a new target, which we named NIS-repressor, for designing therapies to restore radioiodine uptake in disseminated tumors. We mapped its binding-site in the proximal NIS promoter (NIS-repressor binding site; NRBS-P) (Li et al. 2007). This repressor may function in concert with or independent of epigenetic effects on NIS expression via NIS promoter methylation and histone deacetylation (Venkataraman et al. 1999).

The term “promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a nucleotide sequence that can stimulate promoter activity and may be an innate element of the promoter or a heterologous element inserted to enhance the level or tissue-specificity of a promoter. Promoters may be derived in their entirety from a native gene, such as a human gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions. Promoters that cause a nucleic acid fragment to be expressed in most cell types at most times are commonly referred to as “constitutive promoters”.

The term “operably linked” refers to a linkage of polynucleotide elements in a functional relationship. A nucleic acid sequence is “operably linked” when it is placed into a functional relationship with another nucleic acid sequence. For instance, a promoter or repressor is operably linked to a coding sequence if it affects the transcription of the coding sequence. Operably linked means that the nucleotide sequences being linked are typically contiguous. However, some polynucleotide elements may be operably linked, but not directly flanked and may even function in trans from a different allele or chromosome.

The present invention is based, in part, on the identification of a second site in the human sodium-iodine symporter (NIS) promoter region, herein, referred to as NIS-repressor binding site (NRBS-D). We further investigated NIS-repressor by refining NRBS-P, demonstrating sequences at −648 to −620 bp, and an additional NRBS at −987 to −958 bp (NRBS-D; relative to the NIS translation start site) as two core binding sites for NIS-repressor. The homology between NRBS-D and NRBS-P core sequences is 83% in a 23 bp region, with two A/G and two T/C transitions. This constitutes a 23 bp consensus sequence (5′-TG(G/A)GCCT(T/C)A(G/A)TTTCCCCA(T/C)CTGT-3′) (SEQ ID NO. 1) (“consensus NRBS”). NRBS-P and NRBS-D are in opposite orientation in the hNIS promoter and 310 bp apart from each other. A human genome homology search (NCBI/BLAST/blastn suite) shows this consensus sequence to occur (at >90% homology) within two kilobases of the translation start site of 56 different genes, within four kilobases of an additional twenty genes and within seven kilobases of an additional eight genes in the human genome. Among these genes, there are some coding for kinases, receptors, and transporters. A list of genes containing a sequence with >90% homology throughout the entirety of SEQ ID NO. 1 in their promoter regions is shown in Table 1.

EMSA analysis showed proteins in KAK1 nuclear extract that bound to NRBS-P and constitute the NIS-repressor. Electrophoretic analysis of these nuclear extract proteins, UV-crosslinked to the radiolabeled NRBS-P probe, revealed multiple bands, suggesting that NIS-repressor is a protein complex. Several thyroidal transcription factors (Sp1, Ap1, AP2, TTF-1 and Pax8), previously characterized as affecting NIS transcription, were excluded as candidates for NIS-repressor components because double-stranded oligonucleotides containing their respective consensus DNA-binding sites failed to compete against a radiolabeled NRBS-P probe in EMSA analysis.

Unexpectedly, an antibody against human thyroid transcription factor 2 (hTTF-2) (antibody S-18), but not two other anti-TTF-2 antibodies (F-17 or V-20), which recognize different epitopes on TTF-2, altered the migration of the probe-protein complex in supershift assays, demonstrating that human TTF-2 is associated with, or is a part of, the NIS-repressor complex. The three tested antibodies are available from Santa Cruz Biotechnology, Inc. S-18 is an affinity purified goat polyclonal antibody raised against a peptide mapping within an internal region of the human TTF2 polypeptide. The epitope for this antibody is the region from amino acid 100-150 in human TTF2. F-17 is an affinity purified goat polyclonal antibody raised against a peptide mapping within an internal region of human TTF2. The epitope for this antibody is the region from amino acid 140-190 in human TTF2, and S-18 and F-17 do not have competing binding sites. V-20 is an affinity purified goat polyclonal antibody raised against a peptide mapping near the C-terminus of human TTF2.

In one aspect of the invention, an inhibitor of TTF-2 is administered to a patient suffering from thyroid cancer to inhibit the formation of the NIS-repressor complex and/or binding of the NIS repressor to either or both of NRBS-P and NRBS-D and restore iodide uptake in dedifferentiated thyroid carcinoma cells.

Although 5-azacytidine and sodium butyrate have been shown to restore NIS transcription (Venkataraman et al. 1999), these agents did not alter the EMSA pattern using KAK1 nuclear extract, suggesting that NIS-repressor represents a different mechanism of NIS gene regulation. This is consistent with our previous genomic DNase I digestion studies (Li et al. 2007) that failed to demonstrate any effect of these agents on chromatin compaction, suggesting the possibility of non-epigenetic regulatory processes.

The human poly(ADP-ribose) polymerase-1 (PARP-1; EC 2.4.2.30) was identified by proteomic analysis of the nuclear extract from KAK1 cells, as a top candidate for a component of the NIS-repressor complex. PARP-1 was initially known for its role as a DNA-damage sensor, repair and signaling protein. Later studies have shown that PARP-1 also participates in additional critical cellular activities, such as: apoptosis, genetic stability, and gene transcription (Schreiber, et al. 2006). PARP-1 was reported to be able to bind to regulatory sequences by itself (Chiba-Falek, et al. 2005; Zhang, et al. 2002), modify some transcription factors or signal proteins by poly(ADP-ribosyl)ation (Miyamoto, et al. 1999), and influence other protein factors by hetero-complex formation (Simbulan-Rosenthal, et al. 2003). A recent study reveals that PARP-1 has widespread effects upon transcription of diverse genes, either as a positive or negative transcription factor (Krishnakumar, et al. 2008).

ChIP analysis of Cal-62 cells with two commercial anti-PARP-1 antibodies shows that PARP-1 is associated with the NRBS-P region in Cal-62 and KAK1 cells under basal culture conditions without NIS transcription. Furthermore. PJ34, an inhibitor of PARP-1 enzymatic activity (Abdelkarim et al. 2001), effectively stimulated luciferase activity from NIS promoter constructs and also stimulated endogenous hNIS transcription in both KAK1 and Cal-62 cells, confirming that PARP-1 is part of a negative regulatory factor for hNIS gene transcription. Despite the ChIP data indicating that PARP-1 was associated with the hNIS promoter region containing NRBS-P, two different commercial anti-hPARP-1 polyclonal antibodies (that had been effective in the ChIP assay) failed to alter the EMSA pattern on supershift analysis. In addition, two commercial preparations of human PARP-1 failed to produce the same EMSA signals as the nuclear extract from KAK1 cells. It is likely that PARP1 does not directly bind to the NRBS sequence; rather, it is associated with other proteins that contain the critical DNA-binding domain. PJ34 inhibition of PARP1 enzymatic activity may compromise the assembly, stability, or activity of the NIS-repressor protein complex.

In summary, a second core sequence in the human sodium-iodine symporter (hNIS) promoter, NRBS-D, which is a binding site for a trans-active transcriptional repressor, NIS-repressor has been defined. Proteomic analysis revealed PARP-1 as an important constituent of the NIS-repressor protein complex. A known inhibitor of PARP-1 enzymatic activity, PJ34, causes increased endogenous transcription of hNIS in genotypically verified thyroid cancer cells.

In one aspect of the invention there is provided a method of screening for therapeutic agents capable of restoring NIS gene expression and radioiodine uptake in thyroid cancer cells. The method comprises the steps of: i) contacting thyroid cancer cells with a pharmacologic antagonist against one or more components of the NIS repressor protein complex capable of binding to SEQ ID NO. 1, ii) detecting NIS expression or radioiodine uptake by the cell; and iii) selecting the pharmacologic antagonist that results in an increase in NIS expression or radioiodine uptake by the thyroid cancer cells. In certain embodiments the pharmacologic antagonist is an inhibitor of PARP-1 or TTF-2, wherein inhibition thereof comprises inhibition of NIS complex binding to SEQ ID NO. 1 or inhibition of NIS complex formation or function.

The 23 base pair NRBS consensus sequence (SEQ ID NO. 1) may have regulatory importance for multiple diverse human genes. In thyroid oncology, NIS-repressor is a useful target in restoring the effectiveness of radioiodine therapy to dedifferentiated thyroid cancers. In other contexts, the NRBS consensus sequence is a useful target for modifying the expression of one or more of the genes in the human genome to which it is operably linked, some of which appear to play a role in cancer.

In a further aspect of the invention the 23 base pair consensus sequence may be used to screen for compounds or molecules that inhibit or compete with NIS-repressor binding to the consensus sequence (antagonists and agonists of NIS-repressor). In certain embodiments, the consensus sequence is operably linked to a promoter and target gene, e.g., such as a vector which includes the linked elements, and is contacted with a test molecule or compound (or multiple test compounds and/or molecules) under conditions suitable for expression of the target gene. Such assay may also be carried out in the presence of NIS repressor in order to determine those compounds or molecules that may interfere with the activity of the NIS repressor. The effects of such contact on transcription of the target gene are detected via any convenient method, e.g., measurement of a detectable marker encoded by the target gene. The expression of the target gene may be compared to expression of the target gene in the presence of varying amounts of the test compound and/or molecules and/or in the absence of the test compound and/or molecule. Any suitable target gene may be used, such as for example the luciferase gene. The vector may be any suitable plasmid or viral vector, such as an adenovirus vector. One of ordinary skill in the art can construct suitable vectors for use in the present invention.

Alternatively, an electrophoretic mobility shift assay (EMSA) may be used to detect SEQ ID NO. 1-specific binding proteins and/or molecules that bind to SEQ ID NO. 1. Such SEQ ID NO. 1-specific binding proteins may be used to modulate the expression of genes operably linked to SEQ ID NO. 1, such as any of the genes listed in Table 1.

In certain embodiments of the invention, the consensus sequence my be used to select for a “better” NIS-repressor (NIS-repressor agonist) using, for example, the methodology disclosed by Urnov F D, Rebar E J, Biochem Pharmacol. 2002 64(5-6):919-23, which is incorporated herein by reference thereto.

Additional objects and advantages of the disclosure will be set forth in part in the description which follows, and in part will be obvious from the description, or may be learned by practice of the disclosure.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and H (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.).

As used herein “stringent hybridization conditions” are generally selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. High stringency conditions are selected to be equal to the Tₘ point for a particular probe. An extensive guide to the hybridization of nucleic acids is found in Tijssen (1993) Laboratory Techniques in Biochemistry and Molecular Biology, incorporated herein in its entirety.

The following describes materials and methods used in the procedures described in the subsequent Examples.

### EXAMPLES

### Example 1

### A Second NRBS (NRBS-D) in the hNIS Promoter

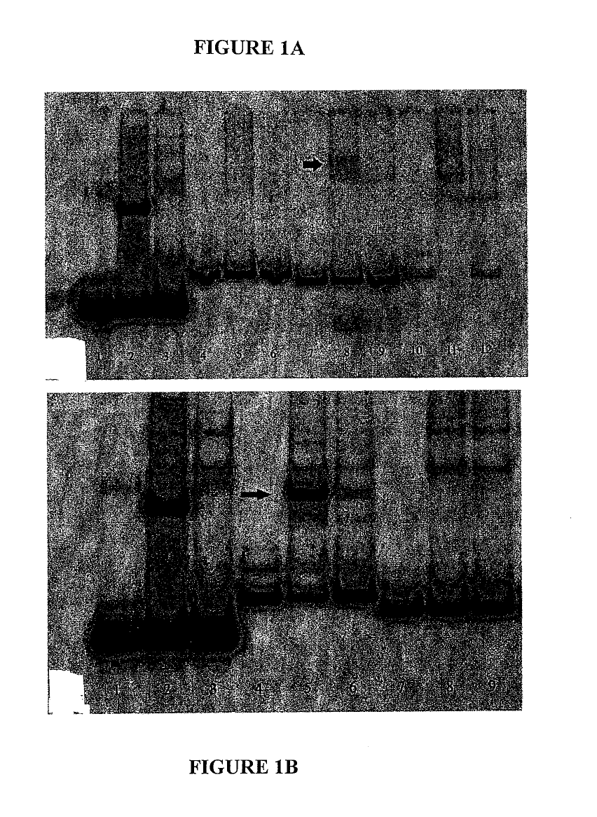

Five EMSA probes, SHIFT-1, -2, -3, -4, and -5, were prepared with PCR, radiolabeled, and used to probe KAK1 nuclear extract in EMSA. The EMSA results shown in FIG. 1 indicate that: 1) no specific signal for SHIFT-1 probe (FIG. 1A, lane 5), covering −1667 to −1468 bp; 2) multiple faint specific signals for SHIFT-2 probe (FIG. 1A, lane 8), covering −1467 to −1268 bp; 3) no specific signal for SHIFT-3 probe (FIG. 1A, lane 11), covering −1267 to −1068 bp; 4) one strong specific signal for SHIFT-4 probe (FIG. 1B, lane 5), covering −1067 to −868 bp; and 5) no specific signal for SHIFT-5 probe (FIG. 1B, lane 8), covering −873 to −708 bp. This shows that KAK1 nuclear extract contains one or more factors that can bind to the sequence from −1067 to −868 bp in the hNIS promoter, further upstream from NRBS-P. We designate this region as a distal NRBS (NRBS-D).

### Example 2

### The Core Sequence of NRBS-D is Homologous to NRBS-P and Demonstrates Cross-Competition Between Both Sites

Seven PCR fragments and three annealed double-strand oligonucleotides were used as unlabeled competitors against the radiolabeled SHIFT-4 probe in EMSA to determine the core sequence for NRBS-D. The seven PCR fragments are: SHIFT-4.1 (150 bp; −1017 to −868), SHIFT-4.2 (100 bp; −967 to −868), SHIFT-4.3 (150 bp; −1067 to −918), SHIFT-4.4 (100 bp; −1017 to −918), SHIFT-4.5 (150 bp; −1017 to −868), SHIFT-4.6 (140 bp; −1007 to −868), and SHIFT-4.7 (130 bp; −997 to −868). The three annealed double-stranded oligonucleotides are: ds-411 (5′-tttattcctctgaggcagggtctattttat-3′, 30 bp; −10.17 to −988) (SEQ ID NO.: 3), ds-412 (5′-tgaggcagggtctattttatccttgttaca-3′, 30 bp; −1007 to −978) (SEQ ID NO.: 4), and ds-413 (5′-tctattttatccttgttacagatggggaaa-3′, 30 bp; −997 to −968) (SEQ ID NO.: 5). Only the sequences of the sense strands are listed. Probe-A and annealed double-stranded Comp-1 were also included as cold competitors, as we considered NRBS-D to be an additional binding site for NIS-repressor, which had already been demonstrated to bind to NRBS-P.

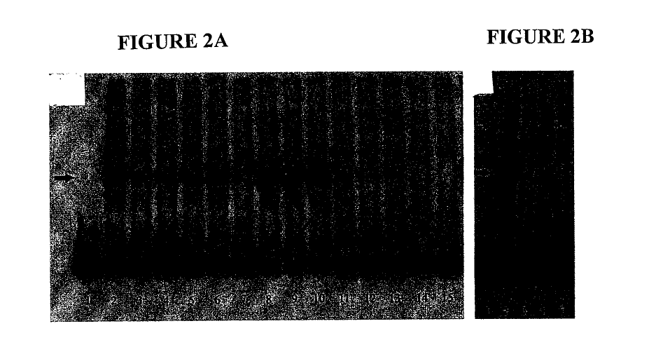

These EMSA results are shown in FIG. 2, revealing that all three annealed double-stranded oligonucleotides (ds-411, ds-412, and ds-413) do not compete against the radiolabeled SHIFT-4 probe (FIG. 2A, lanes 8-10) and that the SHIFT-4.2 fragment does not compete against this probe either (FIG. 2A, lane 6). All of the other PCR fragments (SHIFT-4.1, SHIFT-4.4, SHIFT-4.3, SHIFT-4.5, SHIFT-4.6, and SHIFT-4.7) compete effectively against the radiolabeled SHIFT-4 probe (FIG. 2A, lanes 4, 5, 7, 11-13). The unlabeled Probe-A (FIG. 2A, lane 15) and the unlabeled double-stranded oligonucleotide, Comp-1 (FIG. 2A, lane 14), strongly compete against the same probe. These data suggest that the sequence around −1017 to −968 bp is critical for the effects of NRBS-D and the NIS-repressor binding to NRBS-P can also bind to NRBS-D.

Further analysis, using an unlabeled annealed double-stranded oligonucleotide (ds-414; 5′-ccttgttacagatggggaaactaaggccca-3′, 30 bp; −987 to −958) (SEQ ID NO.: 6), sharing a 20 bp sequence with NRBS-D and having an additional unshared 10 bp sequence downstream, revealed strong competition against the radiolabeled Probe-A in EMSA (FIG. 2B, lane 5). This suggests that the NIS-repressor, binding to NRBS-D, can also bind to the NRBS-P. Thus, NRBS-D and NRBS-P can cross-compete efficiently against each other in EMSA, indicating that NIS-repressor, in KAK1 nuclear extract, can bind to either NRBS-P or NRBS-D in the hNIS promoter region.

### Example 3

### Association of TTF-2 with NRBS-P and NRBS-D

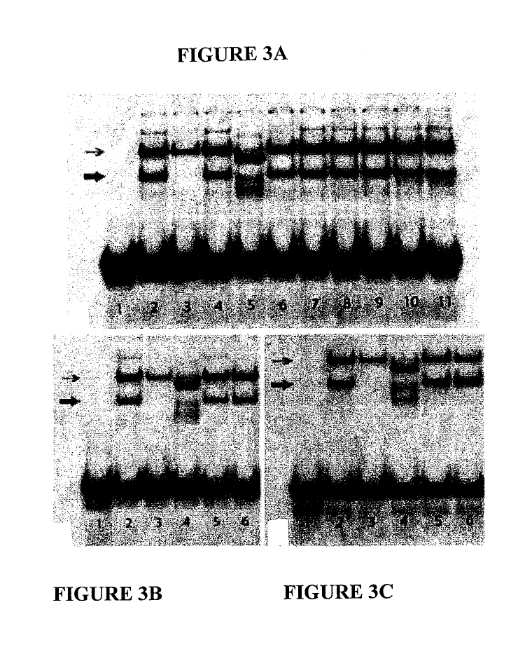

In supershift assays, antibodies against human Sp1 (E-3), c-Jun (H-79), c-Fos (H-125), AP-2α (C-18), TTF-1 (F-12), Pax8 (A-15), and PARP-1 failed to alter the EMSA signal mobilities, suggesting that their respective antigens are not associated with the NRBS-P site. This is consistent with other results showing that their respective consensus DNA target sequences are unable to compete against NRBS-P. The anti-TTF-2 antibody (S-18) shifted the EMSA signals, changing the mobility of one of the bands, showing faster migration, and simultaneously changing the single Comp-1 specific signal into multiple constituent bands with faster migration on the gel, as shown in FIG. 3A, lane 5. We attempted to further verify this phenomenon with two additional anti-TTF-2 commercial antibodies, recognizing different TTF-2 epitopes. Both of these antibodies (F-17, V-20) failed to alter the EMSA signals as achieved with the S-18 antibody. This indicates that TTF-2 is a constituent of the protein factors responsible for the EMSA signals with NRBS-P (FIG. 3B) and NRBS-D probes (FIG. 3C), demonstrating that human TTF-2 is likely to be part of the NIS-repressor complex.

### Example 4

### Identification of the Consensus Sequence in Various Human Genes

A human genome homology search (NCBI/BLAST/blastn suite) using the consensus sequence (5′-TG(G/A)GCCT(T/C)A(G/A)TTTCCC-CA(T/C)CTGT-3′ (SEQ ID NO. 1) was undertaken to determine whether the sequence is present in human genes in addition to the hNIS gene. The consensus sequence is shown to occur (at >90% homology throughout the entire sequence of SEQ ID NO. 1) in the human genes listed in Table 1 below.

| | | Upstream | |
|---|---|---|---|
| | | consensus | |
| | | sequence relative | |
| | | to the translation | |
| | Gene | start codon (bp)of | |
| Gene name | symbol | the gene | Known functions of the gene |
| serine | SDSL | 179 | L-serine, glycine betaine degradation, |
| dehydratase-like | | | isoleucine, leucine, valine biosynthesis |
| perilipin-4 | PLIN4 | 247 | A protein that coats intracellular lipid storage |
| | | | droplets |
| Carboxy | CPZ | 267 | Metal ion binding, metallocarboxypeptidase |
| peptodase Z | | | activity, metallopeptidase activity, peptidase |
| isoform 1 | | | activity, zinc ion binding. |
| small inducible | CCL22 | 334 | Displays chemotactic activity for monocytes, |
| cytokine A22 | | | dendritic cells, natural killer cells and for |
| | | | chronically activated T lymphocytes, also |
| | | | displays a mild activity for primary activated T |
| | | | lymphocytes. Binds to chemokine receptor |
| | | | CCR4. May play a role in the trafficking of |
| | | | activated T lymphocytes to inflammatory sites |
| | | | and other aspects of activated T lymphocyte |
| | | | physiology. |
| orexin receptor 1 | HCRTR1 | 332 | Being a G-protein coupled receptor involved in |
| | | | the regulation of feeding behavior. Selectively |
| | | | binds the hypothalamic neuropeptide orexin A. |
| lethal giant larvae | LLGL1 | 495 | This gene encodes a protein that is similar to a |
| homolog 1 | | | tumor suppressor in Drosophila. The protein is |
| | | | part of a cytoskeletal network and is associated |
| | | | with nonmuscle myosin II heavy chain and a |
| | | | kinase that specifically phosphorylates this |
| | | | protein at serine residues. The gene is located |
| | | | within the Smith-Magenis syndrome region on |
| | | | chromosome 17. |
| | | | Protein binding, protein kinase binding, |
| | | | structural molecule activity, cortical actin |
| | | | cytoskeleton organization, exocytosis, protein |
| | | | complex assembly, maintenance of apical/basal |
| | | | porlarity |
| syndecan-4 | SDC4 | 328 | A transmembrane (type I) heparan sulfate |
| | | | proteoglycan that functions as a receptor in |
| | | | intracellular signaling. Involved in Alpha-actin |
| | | | binding, cytoskeleton protein binding, |
| | | | fibronectin binding, protein kinase C binding, |
| | | | thrombospondin receptor activity |
| ras-related protein | RRAS2 | 329 | A member of the R-Ras subfamily of Ras-like |
| R-Ras2 isoform c | | | small GTPases. Associates with the plasma |
| | | | membrane and may function as a signal |
| | | | transducer. May play an important role in |
| | | | activating signal transduction pathways that |
| | | | control cell proliferation. Mutations in this |
| | | | gene are associated with the growth of certain |
| | | | tumors. Involved in GTP binding, GTPase |
| | | | activity, protein binding, Ras protein signal |
| | | | transduction, positive regulation of cell |
| | | | migration |
| potassium- | ATP4A | 355 | A catalytic alpha subunit of the gastric H+, K+- |
| transporting | | | ATPase. This enzyme is a proton pump that |
| ATPase alpha | | | catalyzes the hydrolysis of ATP coupled with |
| chain 1 | | | the exchange of H(+) and K(+) ions across the |
| | | | plasma membrane. It is also responsible for |
| | | | gastric acid secretion. |
| Transmem-brane | ANO8 | 390 | Chloride channel activity, ion transport |
| protein 16H | | | |
| WSC domain- | WSCD1 | 486 | Acetylglucosaminyltransferase activity, |
| containing protein 1 | | | sulfotransferase activity |
| naked cuticle | NKD1 | 511 | Displays calcium binding, protein binding |
| homolog 1 | | | activity. Involved in Wnt receptor signaling. |
| dipeptidyl | DPP3 | 575 | A member of the M49 family of |
| pepetidase III | | | metallopeptidases. This cytoplasmic protein |
| | | | binds a single zinc ion with its zinc-binding |
| | | | motif (HELLGH) and has post-proline |
| | | | dipeptidyl aminopeptidase activity, cleaving |
| | | | Xaa-Pro dipeptides from the N-termini of |
| | | | proteins. Increased activity of this protein is |
| | | | associated with endometrial and ovarian |
| | | | cancers. |
| diaphanous 2 | PIAPH2 | 546 | Belongs to the diaphanous subfamily of the |
| isoform 156 | | | formin homology family of proteins. This gene |
| | | | may play a role in the development and normal |
| | | | function of the ovaries. Defects in this gene |
| | | | have been linked to premature ovarian failure |
| | | | 2. Involved in Actin binding, receptor binding, |
| | | | oogenesis. |
| DEAH(Asp-Glu- | DHX8 | 590 | With ATP binding, RNA binding, ATP- |
| Ala-His)box | | | dependent RNA helicase activity, protein |
| polypeptide 8 | | | binding, nucleotide binding, hydrolase activity |
| | | | Being a DEAD box protein, which is highly |
| | | | homologous to yeast Prp22. This protein |
| | | | facilitates nuclear export of spliced mRNA by |
| | | | releasing the RNA from the spliceosome. |
| interleukin-10 | IL10 | 524 | A cytokine produced primarily by monocytes |
| | | | and to a lesser extent by lymphocytes; has |
| | | | pleiotropic effects in immunoregulation and |
| | | | inflammation; down-regulates the expression |
| | | | of Th1 cytokines, MHC class II Ags, and |
| | | | costimulatory molecules on macrophages; |
| | | | enhances B cell survival, proliferation, and |
| | | | antibody production; block NF-kappa B |
| | | | activity, and is involved in the regulation of the |
| | | | JAK-STAT signaling pathway. Knockout |
| | | | studies in mice suggested the function of this |
| | | | cytokine as an essential immunoregulator in the |
| | | | intestinal tract. |
| RGS9 anchor | RGS9BP | 593 | A regulator of G protein-coupled receptor |
| protein | | | signaling in phototransduction. Studies in |
| | | | bovine and mouse show that this gene is |
| | | | expressed only in the retina, and is localized in |
| | | | the rod outer segment membranes. This protein |
| | | | is associated with a heterotetrameric complex, |
| | | | specifically interacting with the regulator of G- |
| | | | protein signaling 9, and appears to function as |
| | | | the membrane anchor for the other largely |
| | | | soluble interacting partners. Mutations in this |
| | | | gene are associated with prolonged |
| | | | electroretinal response suppression (PERRS), |
| | | | also known as bradyopsia. |
| nudix (nucleotide | NUDT22 | 619 | |
| diphosphate | | | |
| linked moiety X)- | | | |
| type motif 22 | | | |
| BCL2-antagonist | BAD | 611 | A member of the BCL-2 family regulators of |
| of cell death | | | programmed cell death. This protein positively |
| protein | | | regulates cell apoptosis by forming |
| | | | heterodimers with BCL-xL and BCL-2, and |
| | | | reversing their death repressor activity. |
| | | | Involved in protein binding, protein kinase |
| | | | binding, positive regulation of B and T cell |
| | | | differentiation, phosphatidylinositol-mediated |
| | | | signaling. |
| fractalkine | CX3CL1 | 665 | With chemokine activity, protein binding, |
| (CX3CL1 | | | receptor binding activity. Involved in |
| chemokine ligand | | | angiogenesis in wound healing, cell adhesion, |
| 1) | | | negative regulation of apoptosis, cytokine- |
| | | | mediated signaling pathway, positive |
| | | | regulation of TGF-beta1 production, positive |
| | | | regulation of inflammatory response. |
| myelin gene | C11orf9 | 652 | With DNA-binding, sequencing-specific DNA |
| regulatory factor | | | binding transcription factor activity, involved |
| isoform 2 | | | in cell differentiation, central nervous system |
| | | | myelination, gene regulation, oligodendrocyte |
| | | | development. |
| ADAMTS-like | ADAMTSL5 | 721, 1062 | metalloendopeptidase activity, zinc ion binding |
| protein 5 | | | |
| ATP-binding | ABCC1 | 712 | A member of the MRP subfamily which is |
| cassette, | | | involved in multi-drug resistance. Functions as |
| subfamily C, | | | a multispecific organic anion transporter, with |
| member1 isoform 5 | | | oxidized glutatione, cysteinyl leukotrienes, and |
| | | | activated aflatoxin B1 as substrates. This |
| | | | protein also transports glucuronides and sulfate |
| | | | conjugates of steroid hormones and bile salts. |
| calcium binding | CABP7 | 815 | |
| protein 7 | | | |
| zinc finger protein | ZNF524 | 827 | With DNA-binding, metal ion binding, |
| 524 | | | zinc ion binding activity, involved in gene |
| | | | regulation. |
| FCH domian only | FCHO1 | 833 | A nucleator of clathrin-mediate |
| 1(FCHO1) | | | endocytosis. |
| sulfotransferase | SULT2B1 | 992 | An enzyme that sulfates |
| family, cytosolic, | | | dehydroepiandrosterone but not 4- |
| 2B, member 1 | | | nitrophenol, a typical substrate for the |
| isoform b | | | phenol and estrogen sulfotransferase |
| | | | subfamilies. Having alchol |
| | | | sulfotransferase activity, protein binding, |
| | | | steroid sulfotransferase activity, Involved |
| | | | in steroid metabolic process, sulfate |
| | | | assimilation, xenobiotic metabolic process. |
| sodium-iodide | NIS | 623, 958 | Iodide uptake |
| symporter | | | |
| muscarine | CHRM4 | 945 | Being a G protein-coupled receptors. |
| acetylcholine | | | Influence many effects of acetylcholine in |
| receptor M4 | | | the central and peripheral nervous system. |
| protein | PPP1R14B | 983 | Protein phosphatase inhibitor activity |
| phosphatase | | | |
| regulatory subunit | | | |
| 14B | | | |
| EGF receptor | EPS8L2 | 1016 | A member of the EPS8 gene family, |
| kinase substrate 8 | | | thought to link growth factor stimulation |
| like protein 2 | | | to actin organization, generating functional |
| | | | redundancy in the pathways that regulate |
| | | | actin cytoskeletal remodeling. |
| alpha-actinin-1 | ACTN1 | 1173 | An actin-binding protein with multiple |
| isoform a | | | roles in different cell types. In non-muscle |
| | | | cells, the cytoskeletal isoform is found |
| | | | along microfilament bundles and |
| | | | adherens-type junctions, where it is |
| | | | involved in binding actin to the membrane. |
| | | | In contrast, skeletal, cardiac, and smooth |
| | | | muscle isoforms are localized to the Z-disc |
| | | | and analogous dense bodies, where they |
| | | | help anchor the myofibrillar actin |
| | | | filaments. This gene encodes a nonmuscle, |
| | | | cytoskeletal, alpha actinin isoform and |
| | | | maps to the same site as the structurally |
| | | | similar erythroid beta spectrin gene. |
| fos-related antigen 1 | FOSL1 | 1063 | A leucine zipper proteins that can dimerize |
| | | | with proteins of the JUN family, thereby |
| | | | forming the transcription factor complex |
| | | | AP-1. Displays protein binding, protein |
| | | | dimerization activity, sequenc-specific |
| | | | DNA binding, sequenc-specific DNA |
| | | | binding transcription factor activity. |
| | | | Involved in cellular defense, chemotaxis, |
| | | | female pregnancy, learning, |
| | | | negative/positive regulation of cell |
| | | | proliferation, positive regulation of |
| | | | apoptosis, cell cycle, response to cAMP, |
| | | | response to drugs. |
| platelet-activating | PAFAH2 | 1111 | With 1-alkyl-acetylglycerophosphocholine |
| factor | | | esterase activity, hydrolase activity, |
| acetylhydrolase 2, | | | phospholipid binding; involved in anti- |
| cytoplasmic | | | apoptosis, lipid metabolic process. |
| PI3K, regulatory | PIK3R2 | 1114, 1983 | Contributes to 1-phosphatidylinositol-3- |
| subunit 2(p85- | | | kinase activity; having GTPase activator |
| beta) | | | activity, protein binding, |
| | | | phosphatidylinositol 3-kinase regulator |
| | | | activity; involved in FGF receptor |
| | | | signaling, T cell receptor signaling, insulin |
| | | | receptor signaling, signal transduction, |
| | | | phosphatidylinositol-mediated signaling. |
| arrestin domiain | ARRDC2 | 1174 | |
| containing 2 | | | |
| isoform | | | |
| 2(ARRDC2) | | | |
| non-receptor | TYK2 | 1265 | A member of the tyrosine kinase and, |
| tyrosine-protein | | | more specifically, the Janus kinases |
| kinase TYK2 | | | (JAKs) protein families. This protein |
| | | | associates with the cytoplasmic domain of |
| | | | type I and type II cytokine receptors and |
| | | | promulgate cytokine signals by |
| | | | phosphorylating receptor subunits. It is |
| | | | also component of both the type I and type |
| | | | III interferon signaling pathways. It may |
| | | | play a role in anti-viral immunity. A |
| | | | mutation in this gene has been associated |
| | | | with hyperimmunoglobulin E syndrome |
| | | | (HIES) - a primary immunodeficiency |
| | | | characterized by elevated serum |
| | | | immunoglobulin E. |
| myosin regulatory | MYL9 | 1270 | A myosin light chain that may regulate |
| light chain 9 | | | muscle contraction by modulating the |
| isoform a (MYL9) | | | ATPase activity of myosin heads. The |
| | | | encoded protein binds calcium and is |
| | | | activated by myosin light chain kinase. |
| | | | Having calcium ion binding, motor |
| | | | activity, and is a structural constituent of |
| | | | muscle. |
| proton-coupled | SLC36A2 | 1275 | Displays amino acid transmembrane |
| amino acid | | | transporter activity, hydrogen:amino acid |
| transporter 2 | | | symportyer activity. Involved in ion |
| | | | transport proton tyransport, amino |
| | | | transport, cellular nitrogen compound |
| | | | metabolic process. Mutations in this gene |
| | | | are associated with iminoglycinuria and |
| | | | hyperglycinuria. |
| hyaluronan and | HAPLN4 | 1297 | Binds to hyaluronic acid binding, and |
| proteoglycan link | | | involved in cell adhesion. |
| protein 4 | | | |
| heat shock 27 kDa | HSPB1 | 1313 | Displays identical protein binding, protein |
| protein 1 | | | binding, ubiquitin binding activities, |
| | | | involved in RNA metabolic process, anti- |
| | | | apoptosis, regulation of translational |
| | | | initiation, response to heat, unfolded |
| | | | protein, and virus and in stress resistance |
| | | | and actin organization. Defects in this |
| | | | gene are a cause of Charcot-Marie-Tooth |
| | | | disease type 2F (CMT2F) and distal |
| | | | hereditary motor neuropathy (dHMN). |
| polypyridine tract- | PTBP1 | 1409 | Displays RNA binding, protein binding, |
| bining protein 1 | | | nucleotide binding, poly-pyrimidine tract |
| | | | binding activity. Involved in RNA |
| | | | splicing, mRNA processing, gene |
| | | | expression. |
| Zinc finger | ZNF581 | 1409 | Displays DNA binding, metal ion binding, |
| protein 581 | | | zinc ion binding activity. Involved in |
| | | | transcription regulation. |
| leucine-rich repeat | LGI4 | 1483 | A secreted protein and a member of a |
| LGI family, | | | small family of proteins that are |
| member 4 | | | predominantly expressed in the nervous |
| | | | system. Through binding of axonal |
| | | | Adam22 [a member of the Adam (A |
| | | | disintegrin and metalloprotease) family of |
| | | | transmembrane proteins] to drive the |
| | | | differentiation of Schwann cells. |
| DENN domain- | DENN2D | 1524 | ? |
| containing protein | | | |
| 2D | | | |
| frizzled-8 | FZD8 | 1547 | |
| ADAM | ADAM11 | 1657 | A member of the ADAM (a disintegrin |
| metallopeptidase | | | and metalloprotease) protein family. |
| domain 11 | | | Displays integrin binding, zinc ion |
| preproprotein | | | binding, metallopeptidase activity, |
| | | | metalloendopeptidase activity. Involved in |
| | | | proteolysis, integrin-mediated signaling. |
| glutamate | GRM7 | 1662 | A G-protein coupled receptor. Displays: |
| receptor, | | | G-protein coupled receptor activity, PDZ |
| metabotropic 7 | | | domain binding, adenylate cyclase |
| isoform a | | | inhibitor activity, calcium ion binding, |
| (GRM7) | | | glutamate binding, glutamate receptor |
| | | | activity, serine binding activioty. Involved |
| | | | in synaptic transmission, sensory |
| | | | perception of sound, smell, response to |
| | | | stimulus, negative regulation of cAMP |
| | | | biosynthetic process, adenylate cyclase |
| | | | activity, and glutamate secretion. |
| pre-mRNA- | PRPF19 | 1697 | Being the human homolog of yeast Pso4, a |
| processing factor | | | gene essential for cell survival and DNA |
| 19 | | | repair. Displays DNA binding, identical |
| | | | protein binding, protein binding, ubiquitin- |
| | | | proetin ligase activity, ubiquitin-ubiquitin |
| | | | ligase activity; and involved in DNA |
| | | | reapir, RNA splicing, mRNA precessing, |
| | | | protein polyubiquitination, spliceosome |
| | | | assembly. |
| GATA binding | GATA4 | 1752 | A member of the GATA family of zinc- |
| protein 4 | | | finger transcription factors. Recognize the |
| | | | GATA motif which is present in the |
| | | | promoters of many genes. This protein is |
| | | | thought to regulate genes involved in |
| | | | embryogenesis and in myocardial |
| | | | differentiation and function. Mutations in |
| | | | this gene have been associated with |
| | | | cardiac septal defects. Displays DNA |
| | | | binding, RNA Pol II transcription factor |
| | | | activity, promoter binding, protein |
| | | | binding, sequence-specific DNA binding, |
| | | | zinc ion binding, transcription factor |
| | | | binding activity, and involved in SMAD |
| | | | protein signaling, blood coagulation, cell- |
| | | | cell signaling, in utero embryonic |
| | | | development. |
| forkhead box N4 | FOXN4 | 1778 | Displays DNA bending activity, dsDNA |
| | | | binding, specific RNA pol II transcription |
| | | | factor activity, transcription factor |
| | | | binding, specific transcriptional repressor |
| | | | activity. |
| plexin A1 | PLXNA1 | 1754 | Has receptor activity, semaphoring |
| | | | receptor activity, involved in axon |
| | | | guidance, signaling transduction, |
| | | | multicellular organismal development. |
| frizzled-9 | FZD9 | 1852 | Expressed predominantly in brain, testis, |
| | | | eye, skeletal muscle, and kidney. Displays |
| | | | G-protein coupled receptor activity, PDZ |
| | | | domain binding, Wnt receptor activity, |
| | | | protein homodimereization activity, |
| | | | protein heterodimerization activity. |
| | | | Involved in B cell differentiation, brain |
| | | | development, canonical Wnt receptor |
| | | | signaling, signal transduction, nervous |
| | | | system development, gene regulation. |
| ribosomal protein | RPL36 | 1864 | Displays protein binding activity and is a |
| L36 | | | structural constituent of ribosome. |
| | | | Involved in protein translation and cellular |
| | | | protein metabolic process. |
| lysophospholipase | LYPLA2 | 1961 | Displays hydrolase activity and involved |
| II | | | in fatty acid metabolic process, lipid |
| | | | metabolic process. |
| glumate receptor, | GRIN3B | 1916 | Functions: contribute to calcium channel |
| ionotropic, N- | | | activity, cation channel activity, glycine |
| methyl-D- | | | binding, extracellular-glutamate-gated ion |
| aspartate 3B | | | channel activity, ionotropic glutamate |
| | | | receptor activity, neurotransmitter receptor |
| | | | activity, transporter activity. Involved in |
| | | | ion transport, ionotropic glutamate |
| | | | receptor signaling, regulation of calcium |
| | | | ion transport, protein insertion into |
| | | | membrane. |
| receptor-type | PTPRH | 2013 | A receptor-type protein tyrosine |
| tyrosine-protein | | | phosphatase (PTP), shown to be expressed |
| phosphatase H | | | primarily in brain and liver, and at a lower |
| isoform 2 | | | level in heart and stomach. It was also |
| | | | found to be expressed in several cancer |
| | | | cell lines, but not in the corresponding |
| | | | normal tissues. Displays hydrolase |
| | | | activity, protein binding, transmembrane |
| | | | receptor protein tyrosine phosphatase |
| | | | activity, and involved in apoptosis, and |
| | | | signal transduction, cell growth, |
| | | | differentiation, mitotic cycle, and |
| | | | oncogenic transformation. |
| F-box/LRR-repeat | FBXL16 | 2103 | Displays protein binding activity, involved |
| protein 16 | | | in the SCF complex, a protein-ubiquitin |
| | | | ligase. |
| mps one binder | MOBKL2C | 2263 | The protein encoded by this gene is similar |
| kinase activator- | | | to the yeast Mob1 protein. Yeast Mob1 |
| like 2C | | | binds Mps1p, a protein kinase essential for |
| | | | spindle pole body duplication and mitotic |
| | | | checkpoint regulation. Displays metal ion |
| | | | binding activity. |
| matrix | MMP9 | 2095 | Displays collagen binding, metal ion |
| metalloproteinase-9 | | | binding, protein binding, zinc ion binding, |
| | | | peptidase activity, metalloendopeptidase |
| | | | activity. Involved in skeletal system |
| | | | development, collagen catabolic process, |
| | | | extraccellualr matrix organization, positive |
| | | | regulation of apoptosis. |
| G-CSF-R | CSF3R | 2026 | A member of the family of cytokine |
| | | | receptors. Mutations in this gene are a |
| | | | cause of Kostmann syndrome, also known |
| | | | as severe congenital neutropenia. |
| | | | Functions as a cytokine receptor, and |
| | | | involved in cell surface adhesion and |
| | | | recognization, defense response, signal |
| | | | transduction. |
| 4- | HPD | 2200 | An enzyme in the catabolic pathway of |
| hydroxyphenylpyruvate | | | tyrosine, catalyzing the conversion of 4- |
| dioxygenase | | | hydroxyphenylpyruvate to homogentisate. |
| isoform 2 | | | Defects in this gene are a cause of |
| | | | tyrosinemia type 3 (TYRO3) and |
| | | | hawkinsinuria (HAWK). Displays 4- |
| | | | hydroxyphenylpyruvate dioxygenase |
| | | | activity, metal ion binding, oxidoreductase |
| | | | activity, and involved in tyrosine catabolic |
| | | | process, oxidation-reduction process, |
| | | | cellular nitrogen compound metabolic |
| | | | process, aromatic amino acid family |
| | | | metabolic process. |
| ribosomal protein | RPL36 | 2273 | A ribosomal protein that is a component of |
| L36 | | | the 60S subunit. |
| neuronal PAS | NPAS1 | 2406, 2908 | A member of the basic helix-loop-helix |
| domain protein1 | | | (bHLH)-PAS family of transcription |
| (NPAS1) | | | factors. May play protective or modulatory |
| | | | roles during late embryogenesis and |
| | | | postnatal development. Displays DNA |
| | | | binding, signal transducer activity, |
| | | | transcription regulator activity, sequence- |
| | | | specific DNA binding transcription factor |
| | | | activity. Involved in regulation of gene |
| | | | transcription. |
| tumor necrosis | TNFAIP8L1 | 2415 | ? |
| factor alpha- | | | |
| induced protein 8- | | | |
| like protein 1 | | | |
| synapotodin | SYNPO | 2477 | Displays actin binding, protein binding |
| isoform C | | | activity. Involved in positive regulation of |
| | | | actin filament bundle assembly, regulation |
| | | | of stress fiber assembl, and may play a |
| | | | role in actin-based cell shape and motility. |
| G-protein couple | GPBAR1 | 2495 | A member of the G protein-coupled |
| bile acid receptor 1 | | | receptor (GPCR) superfamily. Functions |
| | | | as a cell surface receptor for bile acids. |
| | | | Treatment of cells expressing this GPCR |
| | | | with bile acids induces the production of |
| | | | intracellular cAMP, activation of a MAP |
| | | | kinase signaling pathway, and |
| | | | internalization of the receptor. The |
| | | | receptor is implicated in the suppression of |
| | | | macrophage functions and regulation of |
| | | | energy homeostasis by bile acids. |
| MRG-binding | C20orf20 | 2643 | Functions: chromatin modification, |
| protein | | | regulation of transcription, regulation of |
| | | | growth. |
| GTP binding | GTPBP3 | 2534 | A GTP-binding protein, localized to the |
| protein 3 | | | mitochondria and may play a role in |
| (mitochondrial) | | | mitochondrial tRNA modification. |
| isoform V | | | Displays GTP binding, GTPase activity, |
| (GTPBP3) | | | nucleotide binding activity. |
| coiled-coil | CCDC102A | 2796 | |
| domain- | | | |
| containing protein | | | |
| 102A | | | |
| intraflagellar | IFT27 | 2966 | A putative GTP-binding protein. Displays |
| transport protein | | | GTP binding, nucleotide binding activity. |
| 27 homolog | | | Involved in small GTPase mediated signal |
| isoform 2 | | | transduction. |
| ALS3 C-terminal- | ALS2CL | 3027 | Displays GTPase activator activity, Rab |
| like protein | | | GTPase binding, Rho guanyl-nucleotide |
| isoform 1 | | | exchange factor activity, identical protein |
| | | | binding activity. Involved in endosome |
| | | | organization, protein localization, |
| | | | regulation of Rho protein signal |
| | | | transduction. |
| interleukin-2 | IL2RB | 3374 | A type I membrane protein and the beta |
| receptor subunit | | | subunit of the interleukin 2 receptor, |
| beta | | | which is involved in T cell-mediated |
| | | | immune responses.. Displays IL-2 receptor |
| | | | activity. Involved in receptor-mediated |
| | | | endocytosis and transduction of mitogenic |
| | | | signals from interleukin 2.signal |
| | | | transduction. |
| protein S100-A8 | S100A8 | 3303 | Displays calcium ion binding, protein |
| | | | binding activity. Involved in the regulation |
| | | | of a number of cellular processes such as |
| | | | cell cycle progression and |
| | | | differentiation. This protein may function |
| | | | in the inhibition of casein kinase and as a |
| | | | cytokine. Altered expression of this |
| | | | protein is associated with the disease |
| | | | cystic fibrosis. |
| rho-related GTP- | RHOD | 3726 | Displays GTP binding, GTPase activity, |
| binding protein | | | nucleotide binding activity. Involved in |
| RhoD | | | endosome dynamics and reorganization of |
| | | | the actin cytoskeleton, and it may |
| | | | coordinate membrane transport with the |
| | | | function of the cytoskeleton. |
| protein kinase C | PACSIN1 | 3998 | Functions: cytoskeletal protein binding, |
| and casein kinase | | | protein kinase activity. Involved in: |
| substrate in | | | endocytosis, cytoskeleton organization. |
| neurons 1 | | | |
| histone-lysine N- | NSD1 | 4074 | Functions: androgen receptor binding, |
| methyltransferase, | | | chromatin binding, estrogen receptor |
| H3 lysine-36, H4 | | | binding, histone methyltransferase activity |
| lysine-20 specific | | | (H3-K36, H4-K20), ligand-dependent |
| | | | nuclear receptor binding, metal ion |
| | | | binding, methylatransferase activity, RAR |
| | | | binding, RXR binding, thyroid hormone |
| | | | receptor binding, transcription cofactor |
| | | | activity, zinc ion binding. Involved in: |
| | | | chromatin modification, regulation of |
| | | | transcription, histone methylation. |
| adenylate kinase | AK1 | 4167 | An enzyme involved in regulating the |
| isoenzyme 1 | | | adenine nucleotide composition within a |
| | | | cell by catalyzing the reversible transfer of |
| | | | phosphate group among adinine |
| | | | nucleotides. Displays: ATP binding, |
| | | | adenylate kinase activity, protein binding, |
| | | | transferase activity, nucleotide kinase |
| | | | activity. Involved in ATP metabolic |
| | | | process, nucleobase, nucleoside and |
| | | | nucleotide metabolic process. |
| ATP-binding | ABCB9 | 4151 | Displays ATP binding, ATPase activity, |
| cassette sub- | | | MHC class I protein binding, TAP1, TAP2 |
| family B membre 9 | | | binding, substrate-specific transmembrane |
| | | | transporter activity, protein |
| | | | homodimerization activity. Involved in |
| | | | multidrug resistance as well as antigen |
| | | | presentation. |
| two pore calcium | TPCN2 | 5581 | A putative cation-selective ion channel |
| channel protein | | | with two repeats of a six-transmembrane- |
| 2(TPCN2) | | | domain. The protein localizes to lysosomal |
| | | | membranes and enables nicotinic acid |
| | | | adenine dinucleotide phosphate (NAADP)- |
| | | | induced calcium ion release from |
| | | | lysosome-related stores. This ubiquitously |
| | | | expressed gene has elevated expression in |
| | | | liver and kidney. Two common |
| | | | nonsynonymous SNPs in this gene |
| | | | strongly associate with blond versus |
| | | | brown hair pigmentation. Displays |
| | | | calcium channel activity, voltage-gated ion |
| | | | channel activity, and involved in |
| | | | transmembrane transport. |
| rho guanine | ARHGEF11 | 6376 | Displays G-protein coupled receptor |
| nucleotide | | | binding, GTPase activator activity, signal |
| exchange factor | | | transducer activity, Rho guanyl-nucleotide |
| 11 isoform 1 | | | exchange factor activity. Involved in |
| | | | signaling, apoptosis, GPCR signaling. |
| | | | Transcription regulation. |
| transcription | SOX10 | 6972 | A member of the SOX (SRY-related |
| factor SOX-10 | | | HMG-box) family of transcription factors |
| | | | involved in the regulation of embryonic |
| | | | development and in the determination of |
| | | | the cell fate. The encoded protein may act |
| | | | as a transcriptional activator after forming |
| | | | a protein complex with other proteins. |
| | | | This protein acts as a nucleocytoplasmic |
| | | | shuttle protein and is important for neural |
| | | | crest and peripheral nervous system |
| | | | development. Mutations in this gene are |
| | | | associated with Waardenburg-Shah and |
| | | | Waardenburg-Hirschsprung disease. |
| | | | Displays DNA binding, RNA pol II |
| | | | transcription factor activity, transcription |
| | | | coactivator activity, sequence-specific |
| | | | DNA binding transcription factor activity. |
| | | | Involved in cell maturation, development, |
| | | | cell differentiation. |
| Histone H1x | H1FX | 7549 | A member of the histone H1 family. |
| | | | Displays DNA binding activity, and |
| | | | involved in nucleosome assembly. |

### Example 5

### Screening for Inhibitors of hNIS Repressor-NRBS Interaction

The electrophoretic mobility shift assay (EMSA) is a simple, rapid, and extremely sensitive method for detecting sequence-specific DNA-binding proteins in crude extracts. Proteins that bind specifically to an end-labeled DNA fragment, (radio-labeled probe) retard the mobility of the fragment during electrophoresis, resulting in discrete band(s) corresponding to the protein-DNA probe complexes. This assay permits the quantitative determination of the affinity, abundance, association and dissociation rate constants, and binding specificity of DNA probe-binding proteins.

Preparation of nuclear extract: Nuclear extracts are prepared from test cells, such as thyroid cells or tumor cells by any acceptable method, such as that encompassed by the NucBuster™ Protein Extraction Kit available from EMD Biosciences Inc./Novagen.

Preparation of Probe: The consensus sequence (SEQ ID NO. 1) is used as probe to detect binding of test molecules/compounds. A polynucleotide comprising the consensus sequence is end-labeled using T4 polynucleotide kinase. Eight pmole annealed double-stranded NRBS probe (consensus sequence), 8 μL γ-P32-ATP (6000 Ci/mmole), 3 μL 10× T4 polynucleotide kinase buffer (New England Biolab) are mixed and distilled water is added to a final reaction volume of 28 μL. Two μL T4 polynucleotide kinase (10000 U/mL (New England BioLab) is added and mixed well. The reaction mixture is incubated at 37° C. for 15 min, unbound label is removed using a QIAquick Nucleotide Removal kit (Qiagen) and the end-labeled probe is eluted with ˜100 μL TE buffer.

EMSA: EMSA is carried out as follows: 3 μL nuclear extract, 1 μL end-labeled probe, 1 μL Poly(dI-dC)-Poly(dI-dC) (0.01 U/μL in 100 mM KCl, 20 mM HEPES, pH 8.0), 1 μL Salmon sperm DNA (500 ng/μL in nuclease-free water), 5 μL 4×EMSA buffer (400 mM KCl, 80 mM HEPES, 0.8 mM EDTA, 80% glycerol, 0.5 mM DTT) are mixed and distilled nuclease-free water is added to bring the reaction mix to 18 μL. The mixture is incubated on ice for 30 minutes, followed by addition of 2 μL of loading buffer (1×EMSA buffer, 0.25% Bromophenol Blue). A 7% non-denaturing PAGE gel is pre-run in 0.5×TBE (5.4 g/L Tris base, 2.75 g/L boric acid, 1 mM EDTA, pH 8.) for 30 minutes at 100V. The entire 20 μL EMSA reaction is loaded into one well of the polyacrylamide gel and run at 100V until the Bromophenol Blue dye has migrated to the end of the gel. The gel is dried on DEAE paper using a standard gel dryer and the dried gel is exposed to X-ray film. A retarded signal relative to free end-labeled probe indicates athe presence of a positive protein-probe interaction complex. When un-labeled DNA probe is added in the EMSA reaction mixture, loss of EMSA signal indicates the signal is probe-specific. When antibody against a specific protein factor is added in the EMSA reaction mixture (Supershift assay), change of EMSA signal indicates that a specific protein factor is involved in the protein-probe complex.

What is claimed is:

1. An isolated nucleic acid sequence comprising the sequence 5′-T/C(G/A)GCCT(T/C)A(G/A)TTTCCCCA(T/C)CTGT-3′ (SEQ ID NO. 1) or a nucleotide sequence that hybridizes to the full length of SEQ ID NO. 1 under high stringency conditions.

2. The isolated nucleic acid sequence of claim 1 wherein said nucleotide sequence shares at least 90% identity throughout the full length of SEQ ID NO. 1.

3. A method of screening for a test molecule or compound that binds to SEQ ID NO. 1, said method comprising (1) contacting the test molecule or compound with a nucleotide sequence comprising the SEQ ID NO. 1 and (2) determining whether the test molecule or compound binds to SEQ ID NO. 1.

4. The method of claim 3 wherein SEQ ID NO. 1 is operably linked to a promoter and a target gene encoding a detectable marker.

5. The method of claim 3 wherein binding of a test compound or molecule to SEQ ID NO. 1 alters expression of the target gene.

6. The method of claim 3 wherein binding of the test molecule or compounds is detected by an electrophoretic mobility shift assay.

7. A method for screening for a test molecule or test compound that interferes with human NIS repressor binding to the human NIS repressor binding site or NIS repressor activity, said method comprising (1) contacting the test molecule or compound in the presence of human NIS repressor with a nucleotide sequence comprising SEQ ID NO. 1 and (2) detecting an alteration in binding of the NIS repressor to SEQ ID NO. 1.

8. The method of claim 7 wherein alteration in binding of NIS repressor to SEQ ID NO. 1 is detected by an electrophoretic mobility shift assay.

9. The method of claim 7 wherein alteration of NIS repressor binding is detected by measuring expression of a target gene operably linked to SEQ ID NO. 1.
